(19) United States
(12) Patent Application Publication
(10) Pub. No.: US 2020/0360305 A1
(43) Pub. Date: Nov. 19, 2020

(54) USE OF TRIETHYLENETETRAMINE (TETA) FOR THE THERAPEUTIC INDUCTION OF AUTOPHAGY

(71) Applicants: INSERM (Institut National de la Ssnté et de la Researche Médecale), Paris (FR); Sorbonne Université, Paris (FR); Université de Paris, Paris (FR); Assistance Publique-Hôpitaux de Paris (APHP), Paris (FR)

(72) Inventors: Guido KROEMER, Paris Cedex 06 (FR); Federico PIETROCOLA, Paris Cedex 06 (FR)

(21) Appl. No.: 16/768,346

(22) PCT Filed: Nov. 30, 2018
(86) PCT No.: PCT/EP2018/083081

§ 371 Date: May 29, 2020

(30) Foreign Application Priority Data
Dec. 1, 2017 (EP) 17306679.6

(51) Int. Cl. A61K 31/132; A61K 45/06; A61P 43/00; A61P 3/04; A61P 3/10; A61P 1/16

(52) U.S. Cl. CPC A61K 31/132; A61K 45/06; A61P 1/16; A61P 3/04; A61P 3/10; A61P 43/00

(57) ABSTRACT

Autophagy is a universal anti-aging mechanism the chronic induction of which can extend the health span and lifespan of mammals Here the inventors show that triethylenetetramine (TETA), also called trientine, a drug that is approved for the treatment of Wilson disease, can induce autophagy in mouse tissues in vivo. In particular, chronic autophagy stimulation by TETA can improve the metabolic characteristics of mice kept on a high-fat or high-sugar diet without reducing their food uptake, yet attenuating their weight gain. TETA attenuates adioposity, signs of obesity related type-2 diabetes and hepatosteatosis. TETA also mediates hepatoprotective effects against acute ethanol intoxication. Hence, TETA can be considered as a novel autophagy-inducing agent and thus can be used for the treatment of various diseases and in particular for the treatment of obesity, as well as obesity-related comorbidities.

### FIELD OF THE INVENTION

The present invention relates to methods and pharmaceutical compositions for inducing autophagy.

### BACKGROUND OF THE INVENTION

Autophagy (“self-eating”) constitutes one of the most spectacular, though subtly regulated phenomena in cell biology and plays a key role in the maintenance of cellular and organismal homeostasis by facilitating the turnover of cytoplasmic structures and allowing cells to adapt to changing and stressful conditions including nutrient deprivation.

The polyamine spermidine is a natural compound endowed with a rather broad health-improving effect, in particular because the molecule induces autophagy. Spermidine supplementation extends the longevity of invertebrate model organisms (1) and mice (2, 3). Moreover, a diet rich in polyamines reduces mortality of aged mice (4). Spermidine administration to mice on a high fat diet (HFD) reduced weigh gain and improved glucose tolerance and insulin sensitivity (5). Moreover, chronic spermidine administration to aging mice protects from cardiac aging. It improves diastolic function, left ventricular elasticity, and mitochondrial function in old mice (2). Spermidine reversed age-induced arterial stiffness with a reduction in oxidative damage of endothelial cells in old mice (6) and alleviated the formation of atherosclerotic plaques in Apolipoprotein E-deficient (ApoE-/-) mice fed a high-fat diet for 20 weeks (7). In Dahl salt-sensitive rats fed a high-salt diet (a model of hypertensive heart failure) oral supplementation of spermidine reduced high blood pressure and delayed the transition to heart failure (2), further documenting the anti-hypertensive (64) and vascular health-promoting (6, 7) effects of dietary spermidine. Beyond these positive effects on cardiometabolic functions, spermidine and other CRMs enhance the anticancer immune response (8), perhaps explaining the capacity of the agent to reduce the incidence of cancers (4). Finally, spermidine protects from age-induced memory impairment (9) and loss of locomotor activity in Drosophila (10). In mice, spermidine attenuates signs of pathology in experimental autoimmune encephalomyelitis, a model for multiple sclerosis (11), blunts retinal degeneration in a model of normal tension glaucoma (12), and improves spatial learning and memory capabilities of old mice (13). Altogether, the current state of the literature indicates that spermidine can be safely used to prevent multiple age-associated pathologies, including metabolic, cardiovascular, neoplastic and neurodegenerative diseases. There is ample evidence that the longevity-extending effects of spermidine, as well as its disease-specific preventive and therapeutic effects are mediated through the capacity of the agent to induce autophagy (2, 14, 15). Accordingly, there is an interest to identify molecule that can mimic the effects spermidine in inducing autophagy.

Triethylenetetramine (TETA), also called trientine, is a clinically used copper-chelating agent. TETA is approved for the treatment of Wilson's disease, a frequent autosomal recessive condition (1 in 30,000 individuals) due to a mutation in the Wilson disease protein (ATP7B), leading to the accumulation of copper within hepatocytes and other cell types (16, 17). Typically, TETA is used for the treatment of patients that have become intolerant to the first-line penicillamine, which is another copper chelator. The typical recommended initial dose of TETA is 500-750 mg/day for pediatric patients and 750-1250 mg/day for adults given in divided doses two, three or four times daily. This may be increased to 2000 mg/day for adults or 1500 mg/day for pediatric patients age 12 or under. Apart from rare allergic reactions, side effects are mild and TETA can be administered to patients for decades, i.e. life-long. Hence its safety prolife is very good. However, the role of TETA in autophagy has not yet been investigated.

### SUMMARY OF THE INVENTION

The present invention relates to methods and pharmaceutical compositions for inducin autophagy. In particular, the present invention is defined by the claims.

### DETAILED DESCRIPTION OF THE INVENTION

Autophagy is a universal anti-aging mechanism the chronic induction of which can extend the health span and lifespan of mammals. Here the inventors show that triethylenetetramine (TETA), also called trientine, a drug that is approved for the treatment of Wilson disease, can induce autophagy in mouse tissues in vivo. These effects are independent from the copper chelating activity of TETA, yet may be related to its capacity to act on polyamine and acetyl coenzyme A metabolism. Chronic autophagy stimulation by TETA can improve the metabolic characteristics of mice kept on a high-fat or high-sugar diet without reducing their food uptake, yet attenuating their weight gain. These effects of TETA extend to mice that are genetically prone to develop obesity even when fed a normal diet. TETA attenuates adioposity, signs of obesity related type-2 diabetes and hepatosteatosis. TETA also mediates hepatoprotective effects against acute ethanol intoxication. In summary, TETA can be considered as a novel autophagy-inducing agent. In particular, TETA can be used as to prevent or treat obesity, as well as obesity-related comorbidities.

The present invention relates to a method of inducing autophagy in a subject in need thereof comprising administering to the subject a therapeutically effective amount of triethylenetetramine (TETA).

As used herein, the term “subject”, “individual,” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

The term “autophagy” refers to macroautophagy, unless stated otherwise, is the catabolic process involving the degradation of a cell's own components; such as, long lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents. The term autophagy may also refer to one of the mechanisms by which a starving cell re-allocates nutrients from unnecessary processes to more essential processes. Also, for example, autophagy may inhibit the progression of some diseases and play a protective role against infection by intracellular pathogens. Acute, intermittent or continuous stimulation of autophagy can delay aging and aging-related diseases including arteriosclerosis, cardiac insufficiency, cancer and neurodegeneration. Stimulation of autophagy can also reduce high-fat or high-sugar diet or high-salt induced weight gain, obesity, metabolic syndrome, hypertension and diabetes.

In some embodiments, the method of the present invention is particularly suitable for inhibiting weight gain. The method is also particularly for reducing glycaemia and lipogenesis. Accordingly, the method of the present invention is particularly suitable for the treatment of various diseases as described herein after.

In some embodiments, the subject is overweight, i.e. a subject having a BMI index superior to 25 kg/m². As used herein, the term “BMI” or “body mass index” has its general meaning in the art and refers to the ratio, which is calculated as body weight per height in meter squared (kg/m²). The BMI provides a simple means of assessing how much an individual's body weight departs from what is normal or desirable for a person of his or her height. Common definitions of BMI categories are as follows: starvation: BMI—less than 15 kg/m²; underweight—BMI less than 18.5 kg/m²; ideal—BMI from 18.5 to 25 kg/m²; overweight—BMI from 25 to 30 kg/m²; obese—BMI from 30 to 40 kg/m²; morbidly obese—BMI greater than 40 kg/m². While simple, the BMI method of characterizing the body weight property of a person is not always correct. For example, the BMI does not take into account factors such as frame size, muscularity or varying proportions of e.g. bone, cartilage, and water weight among individuals. Thus, the accuracy of BMI in relation to actual levels of body fat mass may be distorted by such factors as fitness level, muscle mass, bone structure, gender, and ethnicity. Also, people with short stature and old people tend to have lower BMI values. It is considered, however, that the skilled person, e.g. a physician, will be able to take these factors into account when making the BMI assessment of any given individual.

In particular, the subject is obese. Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m2 to less than 25 kg/m².

Thus, the method of the present invention is particularly suitable for the treatment of obesity and obesity-related disorders. The term “obesity-related disorders” encompasses disorders that are associated with, caused by, or result from obesity. Examples of obesity-related disorders include overeating and bulimia, diabetes, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis, transient ischemic attack, arthritis deformans, sudden death, osteoarthritis, cholelithiasis, gallstones and gallbladder disease, lumbodynia, emmeniopathy, obstructive sleep apnea, stroke, polycystic ovary disease, craniopharyngioma, the Prader-Willi Syndrome, Frohlich's syndrome, GH-deficiency, normal variant short stature, and Turner syndrome. Other examples include pathological conditions showing reduced metabolic activity or a decrease in resting energy expenditure as a percentage of total fat-free mass, such as in children with acute lymphoblastic leukemia. Further examples of obesity-related disorders include metabolic syndrome, insulin resistance syndrome, type II diabete, impaired fasting glucose, impaired glucose tolerance, reproductive hormone abnormalities, sexual and reproductive dysfunction, such as impaired fertility, infertility, hirsutism in females and hypogonadism in males, fetal defects associated with maternal obesity, gastrointestinal motility disorders, such as obesity-related gastro-esophageal reflux, respiratory disorders, such as obesity-hypoventilation syndrome (Pickwickian syndrome), and breathlessness, dermatological disorders, inflammation, such as systemic inflammation of the vasculature, arteriosclerosis, hypercholesterolemia, hyperuricaemia, lower back pain, orthopedic disorders, gout, kidney cancer and increased anesthetic risk, as well as secondary outcomes of obesity such as left ventricular hypertrophy. Obesity-related disorders also include the liver abnormalities associated with obesity such as cirrhosis, liver steatosis, non-alcoholic steatohepatitis (NASH), and non-alcoholic fatty liver disease (NAFLD).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the subject suffers from metabolic syndrome. The term “metabolic syndrome,” as used herein, is present if a person has three or more of the following symptoms: abdominal obesity, hyperglyceridemia, low HDL cholesterol, high blood pressure, and high fasting plasma glucose.

In some embodiments, the subject suffers from type II diabetes. The term “type II diabetes” or “non-insulin dependent diabetes mellitus (NIDDM)” has its general meaning in the art. Type II diabetes often occurs when levels of insulin are normal or even elevated and appears to result from the inability of tissues to respond appropriately to insulin. Most of the Type II diabetics are obese.

In some embodiments, the method of the present invention is particularly suitable for the treatment of cardiometabolic diseases. As used herein, the term “cardiometabolic disease” has its general meaning in the art and relates to cardiovascular diseases associated with metabolic syndrome, such as obesity, diabetes/insulin resistance, hypertension and dyslipidemia. The term “cardiometabolic diseases” refers to cardiac consequences of metabolic syndrome such as atherosclerosis, coronary heart disease, obesity-associated heart disease, insulin resistance-associated heart disease, hypertensive heart disease, cardiac remodeling, heart failure and cardiometabolic diseases disclosed in Hertle et al, 2014; Hua and Nair, 2014; U.S. Pat. Application No. 2012/0214771 and International Patent Application No. 2008/094939. As used herein, the term “age related cardiometabolic disease” relates to any cardiometabolic disease which has a factor of its etiology the age of the subject It will be understood that age may only be one of a number of factors, which combined, result in the development of the disorder.

In some aspects, the method of the present invention is also particularly suitable for the treatment of steatosis. As used herein, the term “steatosis” refers to the condition in which fat accumulates in tissues, such as liver tissue, heart muscle tissue or other muscle tissues. The term “steatosis” does not imply any causative relationship with any metabolic condition or disorder. In some embodiments, the method of the present invention is particularly suitable for the treatment of alcohol-induced hepatic steatosis. In some embodiments, the method of the present invention is particularly suitable for the treatment of non-alcoholic steatohepatitis (NASH). NASH is a progressive disease of the liver of unknown ethiology characterized histologically by fatty acid accumulation, hepatocyte damage and inflammation resembling alcoholic hepatitis. NASH is a critical stage in the process that spans from hepatic steatosis to cirrhosis and liver failure. Obesity and type-2 diabetes are associated to NASH. Since the prevalence of these diseases is increasing, the prevalence of NASH is also expected to increase and therefore, this disease has become an emerging public issue (Reid A E. 2001). More generally, the method the present invention is particularly suitable for the treatment of non-alcoholic fatty liver diseases (NAFLD). NAFLD are disorders with histologic features of alcohol-induced liver disease that occurs in people who do not consume significant amounts of alcohol. Several studies have suggested that this entity is uncommon and that it occurs most often in middle-aged, overweight females. Hyperglycemia with and without evidence of hyperlipidemia is commonly associated with NAFLD and is felt to be a predisposing condition. More recent reports have suggested that NAFLD may be more common than originally suspected and that it may affect individuals who lack the typical risk factors for this disorder.

Thus one object of the present invention relates to a method of treating steatosis in a subject in need thereof comprising administering to the subject a therapeutically effective amount of triethylenetetramine (TETA).

A further object relates to a method of treating alcohol-induced hepatic steatosis in a subject in need thereof comprising administering to the subject a therapeutically effective amount of triethylenetetramine (TETA).

A further object relates to a method of treating non-alcoholic steatohepatitis (NASH) in a subject in need thereof comprising administering to the subject a therapeutically effective amount of triethylenetetramine (TETA).

A further object relates to a method of treating a non-alcoholic fatty liver disease (NAFLD) in a subject in need thereof comprising administering to the subject a therapeutically effective amount of triethylenetetramine (TETA).

In some embodiments, the method of the present invention is particularly suitable for the prevention of liver fibrosis, including cirrhosis. Liver fibrosis is characterized by the accumulation of extracellular matrix that can be distinguished qualitatively from that in normal liver. Left unchecked, hepatic fibrosis progresses to cirrhosis (defined by the presence of encapsulated nodules), liver and organ failure, and death. Chronic liver injury may be the result of chronic alcohol consumption (alcoholic liver disease, steatohepatitis (ASH)), overfeeding, insulin resistance, type 2 diabetes (non-alcoholic fatty liver disease, NASH, steatosis), idiopathic portal hypertension, hepatic fibrosis (including congenital hepatic fibrosis), autoimmune hepatitis, primary sclerosing cholangitis, or primary biliary cirrhosis. In some embodiments, the fibrosis is associated with liver steatosis. The method of the present invention is thus particularly suitable for the prevention of liver cancer that results from liver fibrosis (i.e. cirrhosis).

In some embodiments, the method of the present invention is particularly suitable for the treatment of cancer. Although the underlying mechanism has not been characterized yet, it has been shown that pre-chemotherapy starvation (the most potent autophagy-inducing physiological stimulus able to systemically induce autophagy) significantly increased treatment efficiency and limits the tumour growth. Furthermore, it has been demonstrated that tumours with PI3K over-activation are resistant to dietary restriction, suggesting an important role for autophagy in the chemiosensitization process. This invention might lead to a less aggressive and equivalently effective treatment based on the punctual administration of TETA.

Accordingly, a further object of the present invention relates to a method for treating a cancer in a subject in need thereof comprising administering the subject with a therapeutically effective amount of TETA and a therapeutically effective amount of a chemotherapeutic agent wherein TETA is administered prior to the chemotherapeutic agent.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, TETA is administered 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56h before the administration of the chemotherapeutic agent.

Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine;

mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the method of the present invention is particularly suitable for the treatment of neurodegenerative diseases. Examples of neurodegenerative diseases include but are not limited to Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjögren-Batten disease), Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), MELAS—Mitochondrial Encephalopathy, Lactic Acidosis and Stroke, Multiple System Atrophy, Multiple sclerosis, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Spinocerebellar ataxia (multiple types with varying characteristics), Spinal muscular atrophy, Steele-Richardson-Olszewski disease, Tabes dorsalis, Tay-Sachs Disease, and Toxic encephalopathy. Preferred neurodegenerative diseases include Alzheimer's disease. Neurodegenerative diseases (i.e Alzheimer disease, Parkinson disease, Huntington disease) are a series of different age-dependent or genetic-dependent pathologies, characterized by progressive neuronal death as consequence of accumulation of aggregates of misfolded proteins, damaged organelles, impaired function of cellular clearence mechanisms. Being autophagy a physiological mechanism dedicated to the degradation of potentially harmful and aggregation-prone long-lived proteins, as well as of the recycle of damaged organelles, it is considered as a protective factor against neuronal cell death. In the context of this invention, the treatment of patients with TETA may results in an improvement of the cellular clearance functions and in an amelioration of the symptomatology of different diseases. For example, Huntington disease is a pathology characterized by the progressive expansion of poly-glutamine tail of the protein huntingtin, resulting in its intra-neuronal aggregation. Huntingtin has been demonstrated to be a specific target of the autophagic pathway, and the increase in basal autophagy by administration of TETA can reduce the rate of neuronal death. In two forms of familiar Parkinson disease, recessive mutations in two genes encoding for PINK1 and PARK2, involved in mitophagy, partially account for the pathogenesis of this disease and may render the patients suitable for treatment with TETA. In the same way, autophagy induction may contribute to the removal of alpha-synuclein aggregates (Lewi bodies), responsible for the pathogenesis of sporadic forms of Parkinson disease, most likely due to a saturation of the autophagic system.

In some embodiments, the method of the present invention is particularly suitable for the treatment of infectious diseases. Autophagic process actively participates in a multipronged defense against microorganisms, contributing to their elimination either via the selective delivery of microorganisms to degradative lysosomes (a process referred to as xenophagy) or via the delivery of microbial nucleic acids to endolysosomal compartment (with subsequent activation of innate and adaptive immunity). Clinically relevant pathogens are degraded in vitro by xenophagy; among these, there are bacteria such as group A Streptococcus pyogenes, Mycobacterium tuberculosis, Shigella flexneri, Salmonella enterica, Listeria monocytogenes; viruses such as herpes simplex virus type 1 (HSV 1) and parasites such as Toxoplasma gondii. Moreover in vivo evidences showed that autophagy genes have a protective role against numerous pathogens, including L. monocytogenes, M. tuberculosis, S. enterica, T. gondii, HSV 1. It has been recently shown that the infection mediated by pathogens like Shigella and Salmonella triggers an aminoacids starvation response eventually leading to the elimination of these pathogens via autophagy. Here use of TETA for triggering a pro-autophagic and anti microbial response against bacterial and virus infection may be suitable.

In some embodiments, the method of the present invention is particularly suitable for the treatment of pulmonary emphysema. Mutations in the protein α1-antitrypsin causes pulmonary emphysema, a disease characterized by the accumulation of the aggregated form of the mutant proteins. As for others proteinopathy, autophagy induction by the administration of TETA might ameliorate the symptoms.

In some embodiments, the method of the present invention is particularly suitable for the treatment of cystic fibrosis. A recent pre-clinical study has found as a consequence of a dysfunctional aggrephagy the pathogenicity of cystic fibrosis, due to an impaired clearance of aggregates of the mutant CTFR. Induction of autophagy mediated by administration of an TETA may represent a suitable strategy.

In some embodiments, the method of the present invention is particularly suitable for the treatment of pancreatitis, which is an inflammatory disease of the exocrine pancreas, culminating in a massive necrotic cell death of acinar cells. Although the mechanisms promoting this pathology are still unclear, there is a consensus on the notion that autophagy is impaired in this pathological process. Acinar cells are characterized by large autophagosomes unable to become autophagolysosomes, mainly due to the depletion of lysosomal proteins (i.e. LAMP2). Furthermore, it has been recently shown that loss of Ikka inhibits autophagy flux and promotes the formation of p62-positive protein aggregates, thus contributing to the initiation of the disease. In addition, during the acute phase of the disease, a selective autophagy process called ‘zymophagy’ prevents acinar cells death through degradation of harmful activated zymogen granules. Moreover, TETA, alone or in combination with a lysosomal-targeted therapy, can be suitable for ameliorating the symptomatology of the disease by restoring a normal autophagic flux.

In some embodiments, the method of the present invention is particularly suitable for the treatment of proteinopathies. Inducing autophagy by using TETA may be particularly suitable for the treatment of proteionpathies. Examples of proteinopathies include, but are not limited to Alzheimer's disease, cerebral β-amyloid angiopathy, retinal ganglion cell degeneration, prion diseases (e.g. bovine spongiform encephalopathy, kuru, Creutzfeldt-Jakob disease, variant Creutzfeldt-Jakob disease, Gerstmann-Straussler-Scheinker syndrome, fatal familial insomnia) tauopathies (e.g. frontotemporal dementia, Alzheimer's disease, progressive supranuclear palsy, corticobasal degeration, frontotemporal lobar degeneration), frontemporal lobar degeneration, amyotrophic lateral sclerosis, Huntington's disease, familial British dementia, Familial Danish dementia, hereditary cerebral hemorrhage with amyloidosis (Iclandic), CADASIL, Alexander disease, Seipinopathies, familial amyloidotic neuropathy, senile systemic amyloidosis, serpinopathies, AL amyloidosis, AA amyloidosis, type II diabetes, aortic medial amyloidosis, ApoAI amyloidosis, ApoII amyloidosis, ApoAIV amyloidosis, familial amyloidosis of the Finish type, lysozyme amyloidosis, fibrinogen amyloidosis, dialysis amyloidosis, inclusion body myositis/myopathy, cataracts, medullary thyroid carcinoma, cardiac atrial amyloidosis, pituitary prolactinoma, hereditary lattice corneal dystrophy, cutaneous lichen amyloidosis, corneal lactoferrin amyloidosis, corneal lactoferrin amyloidosis, pulmonary alveolar proteinosis, odontogenic tumor amylois, seminal vesical amyloid, cystic fibrosis, sickle cell disease and critical illness myopathy.

As used herein, the term “TETA” or “Triethylenetetramine” has its general meaning in the art and refers to the N,N′-Bis(2-aminoethyl)ethane-1,2-diamine. The term is also known as Trien; Trientine (INN); or Syprine®(brand name). The term “TETA” encompasses the trientine tetrahydrochloride (4HCL TETA) and the trientine dihydrochloride (2HCL TETA).

By a “therapeutically effective amount” is meant a sufficient amount of the TETA for reaching a therapeutic effect. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 4,000 mg per adult per day. Typically, the compositions contain 0.01, 0.05, 0.1, 0.5, 1.0, 2.5, 5.0, 10.0, 15.0, 25.0, 50.0, 100, 250, 500 and 1000 mg of the active ingredient for the symptomatic adjustment of the dosage to the subject to be treated. A medicament typically contains from about 0.01 mg to about 1000 mg of the active ingredient. An effective amount of the drug is ordinarily supplied at a dosage level from 0.0002 mg/kg to about 50 mg/kg of body weight per day, especially from about 0.001 mg/kg to 10 mg/kg of body weight per day.

TEAT is administered to the subject in a form of a pharmaceutical composition. Typically, the TETA can be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to the subjects. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the present invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. TETA can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Triethylenetetramine (TETA) induces autophagy in vivo. (A, B) Acute administration of TETA (100 mg/kg i.p,) triggers autophagy in wild type C57BL/6 mice heart (A) and liver (B). Autophagic flux was monitored by immunoblotting, following LC3-I to LC3-II conversion (quantified in middle panels) and p62/SQSTM1 degradation (quantified in middle panels) 6 hours after Teta injection. Data represent mean±SEM (n=2 experiments) with n=3 animals/group. **p<0.01 (unpaired t-test). (C, D). TETA treatment reduces protein acetylation in mice heart and liver, as determined by immunofluorescence staining of N-e-acetylated Lysine residues of proteins in cardiomyocytes (C) or hepatocytes (D). Representative images (left) and quantification are shown. Data represent mean±SEM (one representative experiment, n=3) with n=5 animals/group. *p<0.05 (unpaired t-test). Scale bar 50 μm.

FIG. 2. Hepatic heavy metals content is not affected by TETA treatment. Long-term (5 w) administration of TETA (3% w/v, drinking water) does not change hepatic content of bivalent Copper (A), Iron (B) or Zinc (C). Data represent mean±SEM from one representative experiment with n=6 animals/group.

FIG. 3. TETA counteracts weight-gain and signs of metabolic syndrome upon various metabolic challenges. Wild type mice were fed with Chow diet (A), High Fat Diet (B) or 30% sucrose (w/v, drinking water) and treated with TETA, supplied in drinking water (A, B) or per os (100 mg/kg) (C). Weight was monitored at the indicated time points. (D) Leptin-receptor deficient ob/ob mice were treated with TETA and weight gain was followed at the indicated time points. Data (means±SEM) from one representative experiment (n=3) are depicted. ***p<0.001; **p<0.01 (ANOVA for linear modeling). (E-L) Intraperitoneal Glucose Tolerance Test (GTT) and Insulin Tolerance Test (ITT) were performed in Wild type mice fed with Chow diet (E, I), HFD (F, J), Sucrose (G, K) or in ob/ob (H, L) mice treated with TETA as described in Materials and Methods section. Data (means±SEM) from one representative experiment (n=3) are shown. *p<0.05 (unpaired t-test) **p<0.01 (unpaired t-test). (I-L).

FIG. 4. TETA treatment does not affect food intake. Wild Type mice fed with chow (A) or HFD (B) (untreated or treated with TETA) were placed in metabolic cages and food intake was monitored for 5 days. Data represent mean±SEM (one representative experiment, n=3) with n=5 animals/group.

FIG. 5. TETA corrects HFD-induced alterations in plasma adipokine levels. Wild type mice fed with chow diet or HFD were treated with TETA for 8 weeks. Mice serum adipokines levels were detected by ELISA immunoassay. Bars represent loge fold changes (FC)±SD in chow diet fed animals. *p<0.05 (unpaired t-test) **p<0.01 (unpaired t-test); ***p<0.001 (unpaired t-test).

FIG. 6. TETA reduces ethanol-induced hepatic damage. Wild type C57Bl/6 mice were injected with PBS or TETA (100 mg/kg i.p) 30 minutes prior to the administration of 33% (vol/vol) ethanol binge protocol at a total cumulative dosage of 4.5-g/kg by 4 equally divided gavages in 20-minute intervals. (A) Microvescicular steatosis was evaluated through lipid-binding Bodipy® staining in mice liver sections. Representative images (left) and quantification (right) are shown. Data represent means±SEM from one experiment (n=3 animals per group).Scale bar 50 μm. (B, C) Hepatic damage was quantified in mice by monitoring serum Aspartate transaminases (AST) (B) or Alanine transaminases (ALT) (C) activity. Data (mean±SEM) are from one experiment (n=3 animals per group). *p<0.05 (unpaired t-test, Sham vs EtOh) # p<0.05 (unpaired t-test, EtOH vs EtOh+TETA).

FIG. 7. TETA stimulates an acetyl CoA consuming polyamine futile cycle. (A) Representation of liver specific changes in polyamines-related metabolites induced by TETA or Spermidine (Spd) administration to mice fed with Chow Diet (CD) or High Fat Diet (HFD), as detected by metabolomics analysis. Bars stand for loge Fold Change means±SD compared to untreated controls upon Chow diet. (B, C) TETA and Spermidine treatments promote polyamine catabolism and acetyl coenzyme A (AcCoA) depletion. Log2FC of N1-Acetylspermidine/Spd and Acetyl CoA in the liver, heart or muscle of mice treated with TETA or Spermidine upon HFD-regimen. *p<0.05 (unpaired t-test) **p<0.01 (unpaired t-test); ***(unpaired t-test).

FIG. 8: TETA treatment reduces hepatic steatosis induced by high fat diet (HFD). Adult male C57BL/6 mice were fed with HFD for 10 weeks and left untreated or treated with triethylenetetramine (Teta, 3% in drinking water). Histopathological analyses performed in H&E stained specimens revealed that Teta elicited a significant anti-steatosis effect in liver. Data are shown as percentage of steatotic cells (referred to as hepatocytes loaded with lipid droplets, as determined by hematoxylin-eosin staining of paraffin-embedded samples). p<0.01 (Student's t test compared to HFD).

### EXAMPLE

### Methods

Mouse experiments Six-to 7-week-old male WT C57Bl/6 were obtained from Envigo, France. Mice were maintained in specific pathogen-free conditions in a temperature-controlled environment with 12-hr light/12-hr dark cycles and received food and water ad libitum. Animal experiments were in compliance with the EU Directive 63/2010 and protocols 2012_069 and APAFIS #5272-2016042112271931v2 were approved by Ethical Committee of the Gustave Roussy Campus and Cordeliers Research Centre respectively. For weight gain experiments, mice were fed high fat diet (#260HF, Safe, France) or were given 30% sucrose solution in tap water. In all experiments, mice were treated with 3% triethylenetetramine dihydrochloride (TETA) (Sigma Aldrich).

Metal content determination. Copper, Iron and zinc levels in liver homogenates, were analyzed by ICP-OES (Ciros Vision; SPECTRO Analytical Instruments) after wet-ashing of samples with 65% nitric acid (Merck KGaA).

Plasma cytokine measurements. Plasma was harvested from blood collection tubes by centrifugation at 15,000 rpm for 30 min, and stored at −80° C. Cytokines and levels were measured using a mouse serum adipokine immunoassay kit according to the protocol provided by the manufacturer (Cat. # MMHMAG-44K, EMD Millipore, Temecula, Calif., USA).

Quantitative analysis of protein acetylation. Four hours after treatment with TETA, tissues were harvested and fixed with 4% paraformaldehyde solution for at least 4 h, followed by treatment with 15% sucrose (w:v in PBS) for 4 h and with 30% sucrose (w:v in PBS) overnight. Tissue samples were embedded in Tissue-Tek OCT compound (Sakura Finetechnical Co, Ltd) and stored at −80° C. Five micrometer (5 μm) thick tissue sections were prepared with a CM3050 S cryostat (Leica Microsystems), air-dried for 1 h, washed in PBS for 5 min, dried at RT for 30 min, and mounted with VECTASHIELD anti-fading medium. For acetylation staining, 5-μm-thick tissue sections were prepared. Tissues were incubated overnight at 4° C. with anti-Acetyl Lysine antibody (#2551, Cell Signaling) in 2% BSA. After 1 h RT incubation with secondary-HRP conjugated antibody, tissues were mounted with VECTASHIELD anti-fading medium. For measurement, approximately 10 pictures of 5 independent visual fields from at least 3 mice were acquired using an Axio Observer inverted fluorescence microscope equipped with Apotome confocal-like system (Carl Zeiss).

Tissue processing for Immunoblotting. For short-term autophagy induction studies, mice were treated with 100 mg/kg i.p. TETA. 1 to 6 h after TETA administration, mice were sacrificed and tissues were snap-frozen in liquid nitrogen after extraction and homogenized two cycles for 20 s at 5.500 rpm using a Precellys 24 tissue homogenator (Bertin Technologies) in 20 mM Tris buffer (pH 7.4) containing 150 mM NaCl, 1% Triton X-100, 10 mM EDTA and Complete® protease inhibitor cocktail (#000000011873580001, Sigma Aldrich). Tissue extracts were then centrifuged at 12,000 g at 4° C. and supernatants were collected. Protein concentration in the supernatants was evaluated by the bicinchoninic acid technique (#23225, BCA protein assay kit). Protein extracts were run on 4-12% Bis-Tris acrylamide gels (# NP0322, Thermo Fisher Scientific) and electrotransferred to 0.2 μM polyvinylidene fluoride (PVDF) membranes (#1620177,Bio-Rad). Non-specific binding sites were saturated by incubating membranes for 1 h in 0.05% Tween 20 (# P9416, Sigma Aldrich) v:v in Tris-buffered saline (TBS) (# ET220, Euromedex) supplemented with 5% non-fat powdered milk (w:v in TBS), followed by an overnight incubation with primary antibodies specific for LC3B (#2775 Cell Signaling Technology) and SQSTM1/p62 (# H00008878-M0, Abnova). Membranes were cut in order to allow simultaneous detection of different molecular weight proteins. Equal protein loading was monitored by probing membranes with a glyceraldeyde-3-phosphate dehydrogenase (GAPDH)-specific antibody (#2118, Cell Signaling Technology). Membranes were developed with suitable horseradish peroxidase conjugates followed by chemiluminescence-based detection with the Amersham ECL Prime (# RPN2232, GE Healthcare and the ImageQuant LAS 4000 software-assisted imager (GE Healthcare, Piscataway, N.J., USA). Quantification was performed by densitometry by means of Image J software. Autophagy was quantified through evaluation of LC3-II/GAPDH ratio and SQSTM1/GAPDH ratio in accordance to guidelines.

Glucose and Insulin Tolerance Test. For glucose tolerance test (GTT) mice were fasted O/N for 16 hours. 2.5 g/kg/BW glucose solution in PBS was injected intraperitoneally and blood glucose levels was measured by means of glucometer in tail vein blood. Tail snipping was used to obtain blood. Before snips, the tail end was dipped into Bupivicaine (0.25%) for local anesthesia. For the insulin tolerance test (ITT), mice were starved for 4 hours and a solution of 0.75 U/kg/BW insulin was injected intraperitoneally. Blood glucose is measured at 30, 60, and 120 minutes after glucose or insulin injection.

Ethanol ninge experiment. Mice were fasted for 6 hours and then they were administered a 33% (vol/vol) ethanol solution at a total cumulative dosage of 4.5-g/kg by 4 equally divided gavages in 20-minute intervals. Control mice received the same volume of water. Vehicle (DMSO) or DMC were intraperitoneally injected to mice 30 minutes before ethanol administration. Sub-mandibular blood collection occurred 16 hours after ethanol binge. Hepatic damage was quantified as serum ALT (Alanine Aminotransferase activity) and serum AST activity (Aspatate aminotransferase) by means of a specific kit (Alanine Transaminase Activity Assay Kit [ab105134]; Aspartate Aminotransferase Assay Kit [ab105135]). Alternatively, OCT embedded tissues were processed as described above and microvescicular steatosis was evaluated by staining with the lipid-binding Bodipy dye. Bodipy-positive lipid droplets/area of cells were quantified by means of Metamorph (Molecular Devices) software.

Metabolomics analysis. All standard and reagents used were from Sigma Aldrich except Ammonium carbonate (VWR). For sample preparation, about 30 mg of tissues for each condition were first weighted and solubilized into 1.5 mL polypropylene microcentrifuge tubes with ceramic beads with 1 mL of cold lysate buffer (MeOH/Water/Chloroform, 9/1/1, −20° C.). They were then homogenized three times for 20 s at 5500 rpm using a Precellys 24 tissue homogenator (Bertin Technologies, Montigny-le-Bretonneux, France), followed by a centrifugation (10 min at 15000 g, 4° C.). Then upper phase of the supernatant was split in two parts: the first 270 μL used for the gas chromatography coupled to mass spectrometry (GC/MS) experiment in microtubes centrifugation, the others 250 μL used for the ultrahigh pressure liquid chromatography coupled by Mass Spectrometry (UHPLC/MS) experimentations. Concerning the GC-MS aliquots, 150 μL were transferred from the microtube centrifugation to a glass tube and evaporated. 50 μL of methoxyamine (20 mg/mL in pyridine) was added on dried extracts, then stored at room temperature in dark, during 16 hours. The day after, 80 μL of MSTFA was added and final derivatization occurred at 40° C. during 30 minutes. Samples were then transferred in vials and directly injected into GC-MS.Concerning the LC-MS aliquots, the collected supernatant was evaporated in microcentrifuge tubes at 40° C. in a pneumatically-assisted concentrator (Techne DB3, Staffordshire, UK). The LC-MS dried extracts were solubilized with 450 μL of MilliQ water and aliquoted in 3 microcentrifuge tubes (100 μL) for each LC method and one microcentrifuge tube for safety. Aliquots for analysis were transferred in LC vials and injected into LC/MS or kept at −80° C. until injection. For sample preparation of polyamines, about 30 mg of tissues for each condition were first weighted and solubilized into 1.5 mL polypropylene microcentrifuge tubes, with 1 mL of cold lysate buffer with 1% sulfosalicylic acid (MeOH /water 1% SSA, 9/1, −20° C.). They were then homogenized three times for 20 s at 5500 rpm using Precellys 24 tissue homogenator (Bertin Technologies, Montigny-le-Bretonneux, France), followed by a centrifugation (10 min at 15000 g, 4° C.). 600 μL of the upper phase of the supernatant was collected and evaporated in microcentrifuge tubes at 40° C. in a pneumatically assisted concentrator (Techne DB3, Staffordshire, UK). The LC-MS dried extracts were solubilized with 300 μL of MilliQ water, centrifugated (10 min at 15000 g, 4° C.), and 50 μL were transferred in polypropylene vial injection for LC method and the rest was transferred in microcentrifuge tube for safety. Aliquots transferred in polypropylene vials were injected into LC/MS or kept at −80° C. until injection. Targeted analysis of CoAs and nucleoside phosphates by ion pairing ultrahigh performance liquid chromatography (UHPLC) coupled to a Triple Quadrupole (QQQ) mass spectrometer was performed on a RRLC 1260 system (Agilent Technologies, Waldbronn, Germany) coupled to a Triple Quadrupole 6410 (Agilent Technologies) equipped with an electrospray source operating in positive mode. The gas temperature was set to 350° C. with a gas flow of 12 L/min. The capillary voltage was set to 3.5 kV.

10 μL of sample were injected on a Column XDB-C18 (100 mm×2.1 mm particle size 1.8 μm) from Agilent technologies, protected by a guard column XDB-C18 (5 mm×2.1 mm particle size 1.8 μm) and heated at 40° C. by a pelletier oven. Heat the column more than the room temperature allowed rigorous control of the column temperature.

The gradient mobile phase consisted of water with 2 mM of DBAA (A) and acetonitrile (B). The flow rate was set to 0.2 mL/min, and gradient as follow: initial condition was 90% phase A and 10% phase B, maintained during 4 min. Molecules were then eluted using a gradient from 10% to 95% phase B over 3 min. The column was washed using 95% mobile phase B for 3 minutes and equilibrated using 10% mobile phase B for 3 min. The autosampler was kept at 4° C.

At the end of the batch of analysis, column was rinsed with 0.3 mL/min of MilliQ water (phase A) and acetonitrile (phase B) as follow: 10% phase B during 20 minutes, to 90% phase B in 20 minutes, and maintained during 20 minutes before shutdown.

The collision gas was nitrogen. The scan mode used was the MRM for biological samples. Peak detection and integration of the 23 analytes were performed using the Agilent Mass Hunter quantitative software (B.07.01).

The collision gas was nitrogen. The scan mode used was the MRM for biological samples. Peak detection and integration of the 133 analytes were performed using the Agilent Mass Hunter quantitative software (B.07.01).

Targeted analysis of polyamines by ion pairing ultra-high performance liquid chromatography (UHPLC) coupled to a Triple Quadrupole (QQQ) mass spectrometer was performed on a RRLC 1260 system (Agilent Technologies, Waldbronn, Germany) coupled to a Triple Quadrupole 6410 (Agilent Technologies) equipped with an electrospray source operating in positive mode. The gas temperature was set to 350° C. with a gas flow of 12 L/min. The capillary voltage was set to 3.5 kV.

10 μL of sample were injected on a Column Kinetex C18 (150 mm×2.1 mm particle size 2.6 μm) from Phenomenex, protected by a guard column C18 (5 mm×2.1 mm) and heated at 40° C. by a pelletier oven.

The gradient mobile phase consisted of water with 0, 1% of HFBA (A) and acetonitrile with 0.1% of HFBA (B) freshly made. The flow rate was set to 0.2 mL/min, and gradient as follow: initial condition was 95% phase A and 5% phase B. Molecules were then eluted using a gradient from 5% to 40% phase B over 10 min. The column was washed using 90% mobile phase B for 2.5 minutes and equilibrated using 5% mobile phase B for 4 min. The autosampler was kept at 4° C.

At the end of the batch of analysis, column was rinsed with 0.3 mL/min of MilliQ water (phase A) and acetonitrile (phase B) as follow: 10% phase B during 20 minutes, to 90% phase B in 20 minutes, and maintained during 20 minutes before shutdown.

The collision gas was nitrogen. The scan mode used was the MRM for biological samples. Peak detection and integration of the 14 analytes were performed using the Agilent Mass Hunter quantitative software (B.07.01). Peak detection and integration were performed using the Thermo Xcalibur quantitative software.

### Results

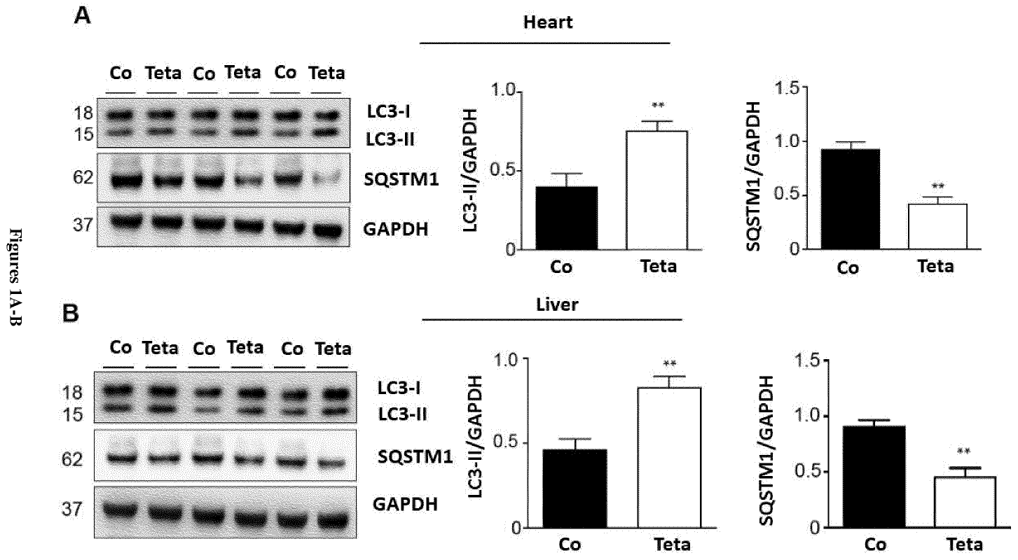

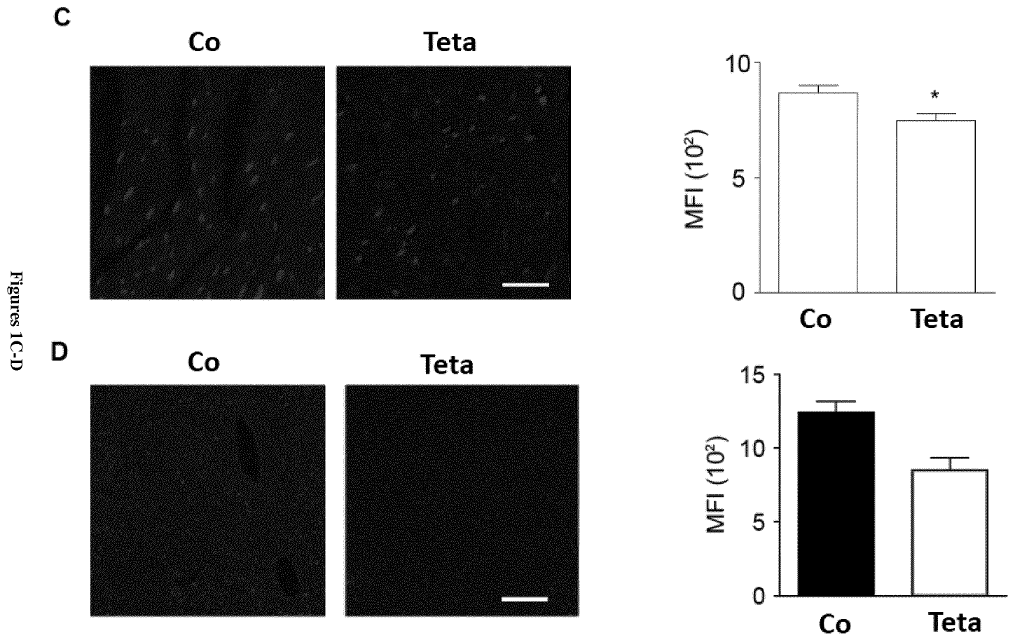

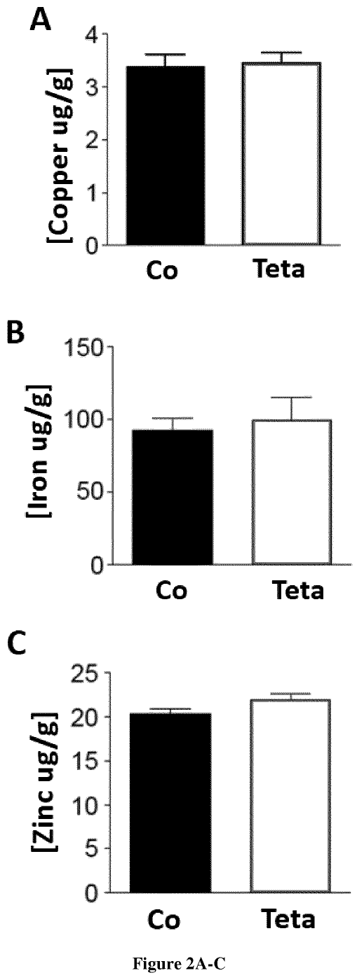

Autophagy induction by triethylenetetramine (TETA) in vivo. Systemic injection of TETA (FIG. 1) or oral administration of the compound (not shown) induced signs of autophagy in several mouse organs, as exemplified for heart (FIG. 1A) and the liver (FIG. 1B), in which Microtubule-associated proteins 1A/1B light chain 3B (hereafter referred to as LC3) exhibited an increase in electrophoretic mobility. This effect was accompanied by a reduction in protein acetylation (FIG. 1C, D). Even long-term administration (5 weeks) of high doses of TETA (3% weight/volume in the drinking water), however, failed to reduce the copper content of organs including the liver (FIG. 2A). Similarly, other heavy metals such as iron (FIG. 2B) and zinc (FIG. 2C) were not affected by TETA. Hence, the pro-autophagic effect of TETA is not mediated by copper depletion but must involve other mechanisms.

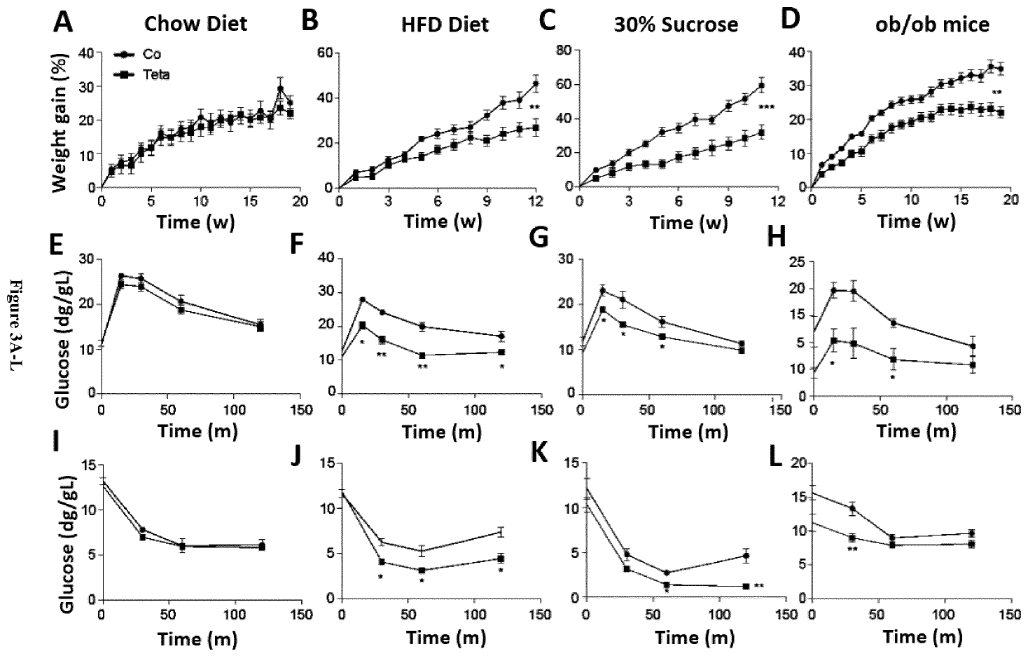

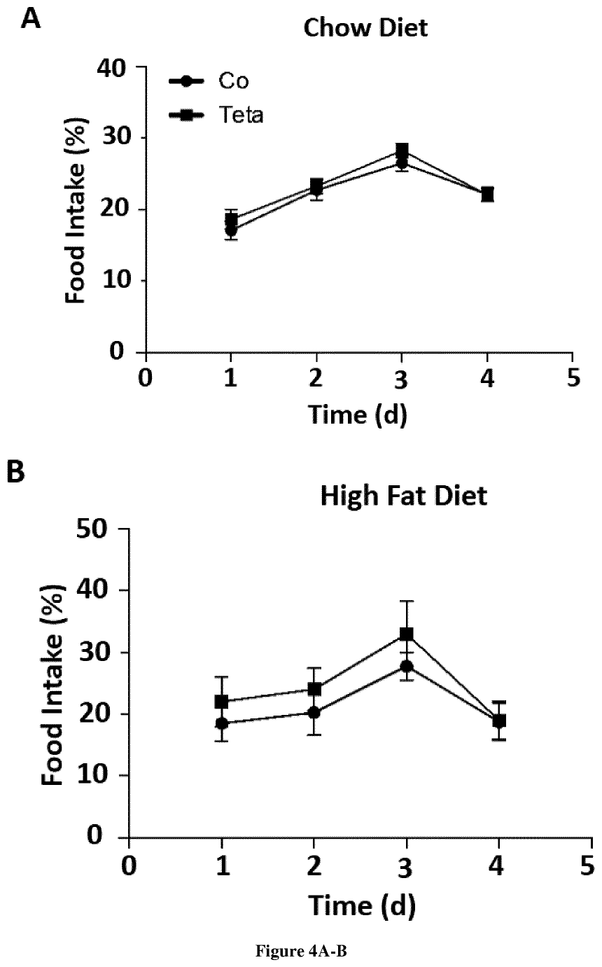

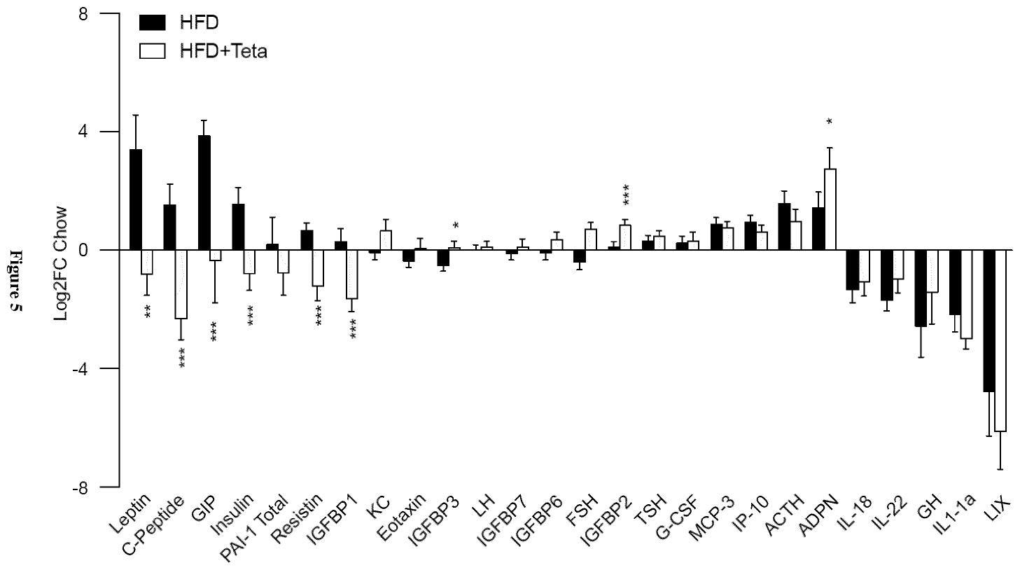

TETA prevents weight gain and diabetes induced by high-fat diet, high-sucrose diet or genetically determined hyperphagy. Although continuous administration of TETA with the drinking water or by gavage failed to affect the physiological weight gain of mice kept on a standard chow (FIG. 3A), it reduced the accelerated weight gain of normal mice on a high-fat diet (FIG. 3B) or on a high-sucrose diet (FIG. 3C), as well as that of mice that overeat due to a genetic defect in the leptin receptor (ob/ob mice) (FIG. 3D). While chronic treatment with TETA had no effect on the glucose-tolerance test and insulin tolerance test of WT mice fed a normal diet, it reduced the plasma glucose concentration after intraperitoneal administration of glucose (glucose tolerance test, GTT, FIG. 3E-H) and enhanced the transient decrease in glycemia after injection of insulin (insulin tolerance test, ITT, FIG. 3I-L) associated with excessive weight gain. This effect was associated with a reduced accumulation of intestinal and perigonadal adipose tissue and reduced fat accumulation in the liver (not shown). Importantly, TETA did not affect food uptake in mice fed a normal diet (FIG. 4A) or a high fat diet (FIG. 4B). In contrast, TETA prevented the HFD-induced elevation of plasma leptin, gastric inhibitory peptide (GIP), C-peptide, insulin, plasminogen activator inhibitor-1 (PAI-1), resistin and IGFBP1, as it increased the adiponectin levels (FIG. 5).

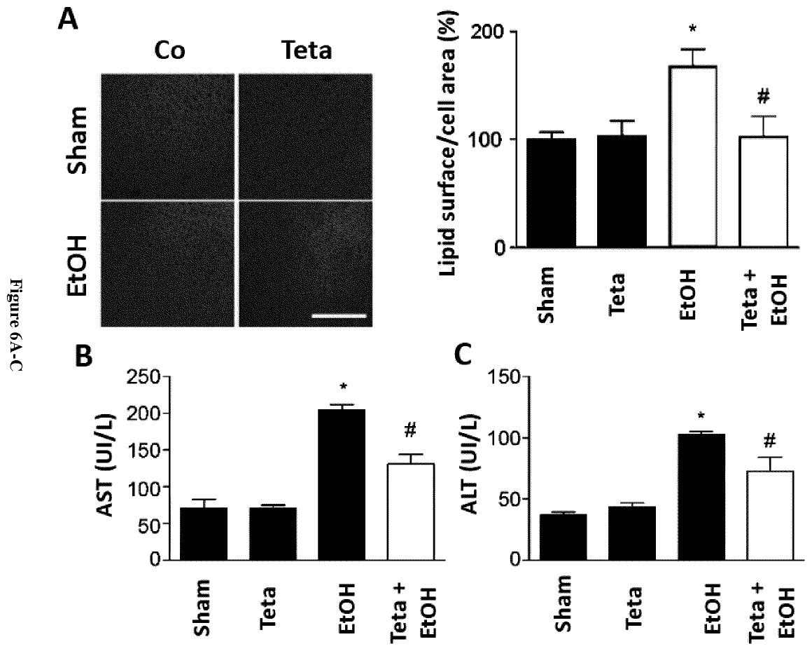

TETA counteracts ethanol-induced hepatic damage. The aforementioned data indicate that TETA can reduce fat accumulation in the liver (hepatic steatosis), which is one of the first steps in the cascade linking obesity to non-alcoholic steatohepatitis. We also investigated whether TETA might counteract acute ethanol-induced liver damage. For this, TETA or vehicle was administered to mice prior to acute ethanol administration, followed by assessment of liver damage by measuring microsteatosis (by Bodipy staining of lipid vesicles in hepatocytes, FIG. 6A) or enzymatic methods (by measuring the plasma concentration of aspartate transaminases (AST) and alanine transaminase (ALT) (FIG. 6B). TETA was able to reduce both the histological and enzymatic signs of acute ethanol-induced liver damage (FIG. 6).

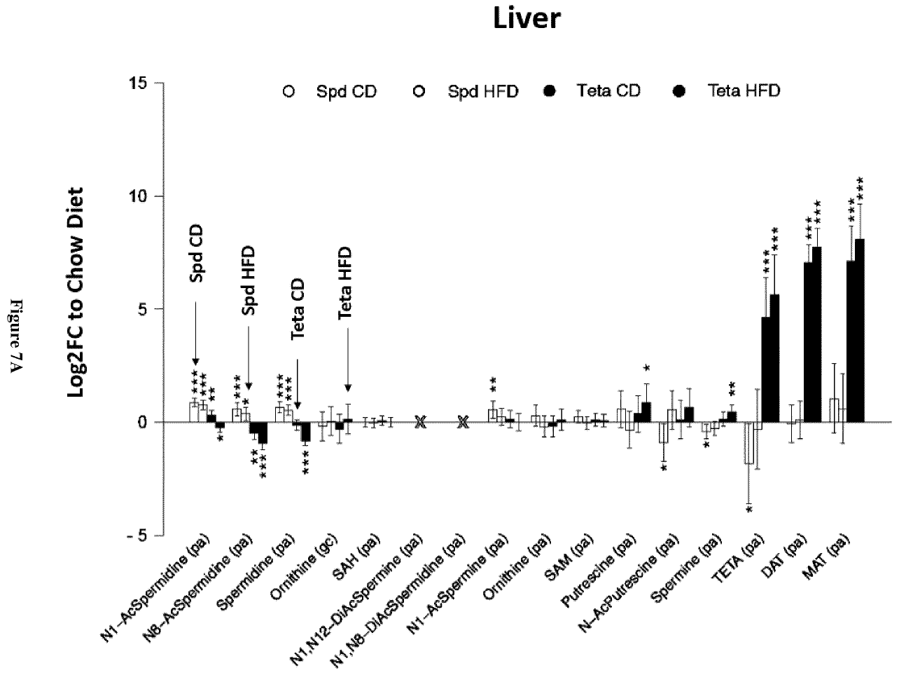

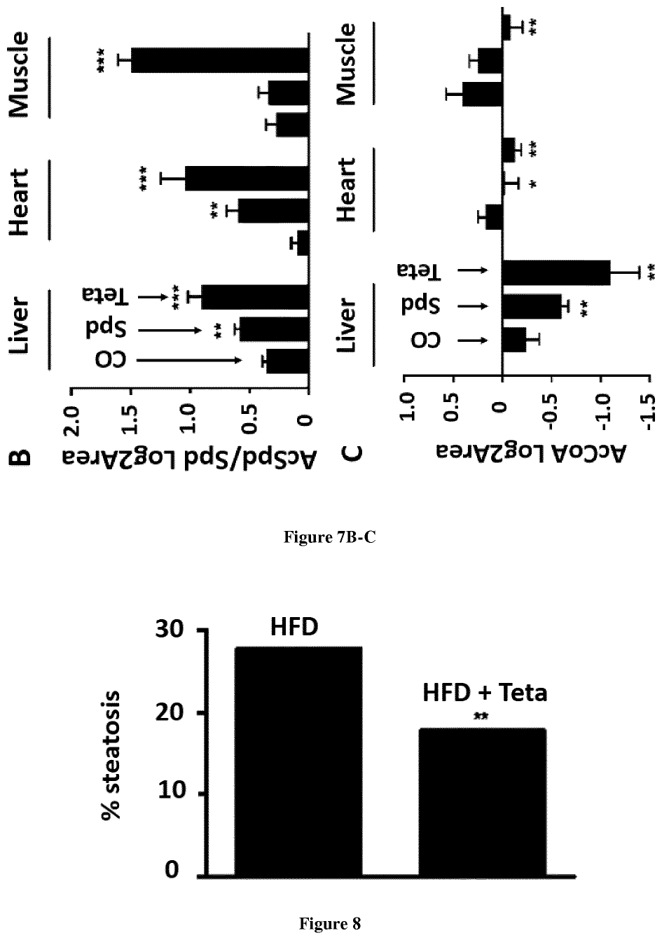

Metabolism and mode of action of TETA. The administration of TETA or spermidine to mice fed a normal or high-fat diet was followed by mass spectrometric metabolomics analyses to determine the metabolic products of these polyamines. TETA yielded two acetylated metabolites, namely Ni-acetyltriethylenetetramine (MAT) and N₁,N₁₀-diacetyltriethylenetetramine that became detectable in all mouse organs (data not shown), similar to prior results obtained in patients (18, 19). Spermidine administration resulted in an increase of this polyamine in most organs except plasma, as well as an increase in N₁-acetylspermidine levels in the liver (FIG. 7A) and in all mouse organs (data not shown). Both TETA and spermidine similarly increased the ratio of Ni-acetylspermidine over spermidine (FIG. 7B), and both of them reduced acetyl coenzyme A (CoA) levels in the liver (FIG. 7C). These results suggest that TETA might increase the activity of spermidine/spermine acetyltransferases 1 (SSAT1), which catalyzes the transfer of acetyl groups form acetyl CoA to spermidine (or spermine) to generate Ni-acetylspermidine (or N₁-acetylspermine).

TETA reduces hepatic steatosis induced by high fat diet (HFD). The mice were fed with high fat diet and were treated or not with TETA. The results revealed that TETA elicited a significant anti-steatosis effect in liver (FIG. 8).

### CONCLUSIONS

The present study reveals the capacity of TETA to mediate a series of effects that are common with those with spermidine, at several levels. First, TETA induces autophagy as well as cytoplasmic protein deacetylation, which are the two most important effects of caloric restriction mimetics, which are non-toxic, anti-aging compounds that induce autophagy as a result of protein deacetylation reactions (20). Second, TETA has the capacity to reduce weight gain, adiposity and hepatosteatosis in several mouse models of obesity with distinct etiologies, namely high-fat diet, high-sucrose diet and genetically determined hyperphagy. Third, as does spermidine, TETA can prevent signs of obesity-associated type-2 diabetes including hyperinsulinemia. This latter effect extends to additional endocrine features of high-fat diet-induced obesity that are corrected by TETA. Fourth, as spermidine does, TETA can mediate cytoprotective functions in vivo, as exemplified for ethanol-induced acute liver damage. Fifth, TETA reduces hepatic steatosis induced by high fat diet. These communalities suggest that TETA may essentially mediate all the health-improving and anti-aging effects of spermidine.

### REFERENCES

Throughout this application, various references describe the state of the art to which this invention pertains. The disclosures of these references are hereby incorporated by reference into the present disclosure.

1. T. Eisenberg et al., Induction of autophagy by spermidine promotes longevity. Nat Cell Biol 11, 1305-1314 (2009).

2. T. Eisenberg et al., Cardioprotection and lifespan extension by the natural polyamine spermidine. Nat Med 22, 1428-1438 (2016).

3. F. Yue et al., Spermidine Prolongs Lifespan and Prevents Liver Fibrosis and Hepatocellular Carcinoma by Activating MAP1S-Mediated Autophagy. Cancer Res 77, 2938-2951 (2017).

4. K. Soda, Y. Dobashi, Y. Kano, S. Tsujinaka, F. Konishi, Polyamine-rich food decreases age-associated pathology and mortality in aged mice. Exp Gerontol 44, 727-732 (2009).

5. A. F. Fernandez et al., Autophagy couteracts weight gain, lipotoxicity and pancreatic beta-cell death upon hypercaloric pro-diabetic regimens. Cell Death Dis 8, e2970 (2017).

6. T. J. LaRocca, R. A. Gioscia-Ryan, C. M. Hearon, Jr., D. R. Seals, The autophagy enhancer spermidine reverses arterial aging. Mech Ageing Dev 134, 314-320 (2013).

7. C. F. Michiels, A. Kurdi, J. P. Timmermans, G. R. Y. De Meyer, W. Martinet, Spermidine reduces lipid accumulation and necrotic core formation in atherosclerotic plaques via induction of autophagy. Atherosclerosis 251, 319-327 (2016).

8. F. Pietrocola et al., Caloric Restriction Mimetics Enhance Anticancer Immunosurveillance. Cancer Cell 30, 147-160 (2016).

9. V. K. Gupta et al., Restoring polyamines protects from age-induced memory impairment in an autophagy-dependent manner. Nat Neurosci 16, 1453-1460 (2013).

10. N. Minois, P. Rockenfeller, T. K. Smith, D. Carmona-Gutierrez, Spermidine feeding decreases age-related locomotor activity loss and induces changes in lipid composition. PLoS One 9, e102435 (2014).

11. Q. Yang et al., Spermidine alleviates experimental autoimmune encephalomyelitis through inducing inhibitory macrophages. Cell Death Differ 23, 1850-1861 (2016).

12. T. Noro et al., Spermidine Ameliorates Neurodegeneration in a Mouse Model of Normal Tension Glaucoma. Invest Ophthalmol Vis Sci 56, 5012-5019 (2015).

13. R. Kibe et al., Upregulation of colonic luminal polyamines produced by intestinal microbiota delays senescence in mice. Sci Rep 4, 4548 (2014).

14. E. Morselli et al., Spermidine and resveratrol induce autophagy by distinct pathways converging on the acetylproteome. J Cell Biol 192, 615-629 (2011).

15. F. Pietrocola et al., Spermidine induces autophagy by inhibiting the acetyltransferase EP300. Cell Death Differ 22, 509-516 (2015).

16. G. J. Brewer, Practical recommendations and new therapies for Wilson's disease. Drugs 50, 240-249 (1995).

17. M. Patil, K. A. Sheth, A. C. Krishnamurthy, H. Devarbhavi, A review and current perspective on Wilson disease. J Clin Exp Hepatol 3, 321-336 (2013).

18. J. Lu, Y. K. Chan, S. D. Poppitt, G. J. Cooper, Determination of triethylenetetramine (TETA) and its metabolites in human plasma and urine by liquid chromatography-mass spectrometry (LC-MS). J Chromatogr B Analyt Technol Biomed Life Sci 859, 62-68 (2007).

19. J. Lu et al., Pharmacokinetics, pharmacodynamics, and metabolism of triethylenetetramine in healthy human participants: an open-label trial. J Clin Pharmacol 50, 647-658 (2010).

20. F. Madeo, F. Pietrocola, T. Eisenberg, G. Kroemer, Caloric restriction mimetics: towards a molecular definition. Nat Rev Drug Discov 13, 727-740 (2014).

1. A method of inducing autophagy in a subject in need thereof comprising
administering to the subject a therapeutically effective amount of triethylenetetramine (TETA).

2. The method of claim 1 wherein the subject is overweight.

3. The method of claim 1 wherein the subject suffers from obesity.

4. A method of inhibiting weight gain and/or reducing glycaemia and lipogenesis in a subject in need thereof, comprising
administering to the subject a therapeutically effective amount of triethylenetetramine (TETA).

5. A method of treating steatosis or non-alcoholic fatty liver disease (NAFLD) in a subject in need thereof comprising administering to the subject a therapeutically effective amount of triethylenetetramine (TETA).

6. The method of claim 5, wherein the steatosis is alcohol-induced hepatic steatosis.

7. The method of claim 5, wherein the steatosis is non-alcoholic steatohepatitis (NASH).

8. (canceled)

9. The method of claim 1 wherein the subject suffers from an obesity related disorder selected from the group consisting of overeating and bulimia, diabetes, hypertension, elevated plasma insulin concentrations and insulin resistance, dyslipidemia, hyperlipidemia, heart disease, cardiovascular disorders, abnormal heart rhythms and arrhythmias, myocardial infarction, congestive heart failure, coronary heart disease, angina pectoris, cerebral infarction, cerebral thrombosis, transient ischemic attack, arthritis deformans, sudden death, osteoarthritis, cholelithiasis, gallstones and gallbladder disease, lumbodynia, emmeniopathy, obstructive sleep apnea, stroke, polycystic ovary disease, craniopharyngioma, the Prader-Willi Syndrome, Frohlich's syndrome, GH-deficiency, normal variant short stature, and Turner syndrome.

10. The method of claim 1 wherein the subject suffers from metabolic syndrome or type II diabetes.

11. The method of claim 1 wherein the subject suffers from steatosis.

12. The method of claim 1 wherein the subject suffers from alcohol-induced hepatic steatosis or non-alcoholic steatohepatitis (NASH).

13. The method of claim 1 wherein the subject suffers from a non-alcoholic fatty liver disease.

14. The method of claim 1 wherein the subject suffers from cancer.

15. The method of claim 14 further comprising administering to the subject a therapeutically effective amount of a chemotherapeutic agent, wherein the TETA is administered prior to the chemotherapeutic agent.

16. The method of claim 14 wherein the cancer is selected from the group consisting of giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lympho sarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

17. The method of claim 1 wherein the subject suffers from a neurodegenerative disease selected from the group consisting of Adrenoleukodystrophy (ALD), Alexander's disease, Alper's disease, Alzheimer's disease, Amyotrophic lateral sclerosis (Lou Gehrig's Disease), Ataxia telangiectasia, Batten disease, Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Mitochondrial Encephalopathy, Lactic Acidosis and Stroke, Multiple System Atrophy, Multiple sclerosis, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Spinocerebellar ataxia, Spinal muscular atrophy, Steele-Richardson-Olszewski disease, Tabes dorsalis, Tay-Sachs Disease, and Toxic encephalopathy.

18. The method of claim 1 wherein the subject suffers from an infectious disease, pulmonary emphysema, cystic fibrosis, pancreatitis, or a proteinopathy.
